(19) United States
(12) Patent Application Publication
(10) Pub. No.: US 2006/0200060 A1
(43) Pub. Date: Sep. 7, 2006

(54) TRACTION DEVICE WITH DIAPHRAGM AND CONNECTION THERETO

(75) Inventors: Christian HUNT, McLean, VA (US); Ara BOGHOSIAN, Alexandria, VA (US); John A. SPADAVECCHIA, Red House, VA (US)

Correspondence Address: Christian Hunt;C/O Josh Witmer, Care Rehab & Orthopedic Products, Inc., P. O. Box 580, McLean, VA, 22101, US

(73) Assignee: CARE REHAB & ORTHOPEDIC PRODUCTS, INC., McLean, VA (US)

(21) Appl. No.: 11/275,970

(22) Filed: Feb. 7, 2006

Related U.S. Application Data
(60) Provisional application No. 60/653,106, filed on Feb. 16, 2005.

(51) Int. Cl. A61F 5/00

(52) U.S. Cl. 602/32; 602/36

(57) ABSTRACT

A device includes a stationary housing. A carriage is slidable mounted within the housing and at least one corrugated diaphragm mechanism is housed in the housing and connectable to the carriage. The corrugated diaphragm is structured to elastically expand and retract, depending on an application of pressure, to extend and retract, respectively, the carriage relative to the stationary housing. A connecting assembly is provided to connect the corrugated diaphragm to the carriage.

### CROSS REFERENCE TO RELATED APPLICATIONS

This application claims priority to U.S. Provisional Application Ser. No. 60/653,106, filed on Feb. 16, 2005, the contents of which are incorporated in its entirety herein. This application is also related to co-pending U.S. application Ser. No. ______, filed on ______ (Attorney Docket No. 26866) which also claims priority to U.S. Provisional Application Ser. No. 60/653,106, filed on Feb. 16, 2005, the contents of which are incorporated in its entirety herein.

### FIELD OF THE INVENTION

The invention generally relates to a cervical and/or lumbar traction device, and more particularly, to a cervical and/or lumbar traction device having a diaphragm with a connection thereto.

### DISCUSSION OF BACKGROUND INFORMATION

Traction devices are used to relieve pressure on inflamed or enlarged nerves. Cervical and lumbar or spinal traction devices are the most common type of these devices. When correctly used, the traction devices can relieve pain in the neck and the spine by, for example, straightening the curvature of the spine or stretching of the spinal and cervical musculature.

Portable traction devices are now becoming very popular for in home use. These devices allow patients to perform traction therapy without leaving their homes, or expending large sums of money for a healthcare provider or physical therapist. Under the proper guidance and instruction, these portable devices are becoming ever more common, especially in today's age of rising health care costs.

Additionally, known portable lumbar traction devices, currently manufactured and sold, are cumbersome and difficult to transport. These lumbar traction devices are basically a large board of approximately 3 or more feet in length.

In one known portable lumbar traction device, two flat separate boards are assembled in order to form the platform for the device. To make such assembly, hooks extending from one flat board are aligned with holes in the other flat board. This is accomplished, most typically, by lifting and aligning the board with the hooks and then inserting the hooks into the holes. During this assembly, a piston rod of a pneumatic device remains attached to one board and the cylinder housing of the pneumatic cylinder remains attached to the other board, making for a very awkward assembly. To disassemble the traction device, the hooks must be removed from the holes, which is an awkward process, especially in view of the piston rods and cylinders remaining attached between the separated boards.

### SUMMARY OF THE INVENTION

In a first aspect of the invention, the device includes a frame and a carriage slidable mounted on the frame. At least one diaphragm is connectable to the carriage by a mounting carriage assembly. The at least one diaphragm is structured to elastically expand and retract, depending on an application of pressure, respectively, and to move the carriage along the frame. The mounting carriage assembly mounts to the carriage.

In another aspect of the invention, the device includes a frame for accommodating at least one corrugated diaphragm device. The at least one corrugated diaphragm device has a retractable spring force such that, upon release of pressure within the at least one corrugated diaphragm device, the at least one corrugated diaphragm device retracts the moveable carriage towards an initial position. The connecting assembly includes at least a connecting disk connectable between a body of the connecting assembly and the at least one corrugated diaphragm device and a mounting structure for connecting to the moveable carriage.

In another aspect of the invention, a device comprises a mounting carriage assembly comprising a frame having at least one mounting connection and at least one end plate. The at least one end plate includes opposing flanges. A connecting disk is mountable between the opposing flanges. The connecting disk includes a slot having a shoulder.

In yet another aspect of the invention, a device comprises a mounting carriage assembly which includes a frame having at least one mounting connection and at least one end plate. The at least one end plate has a slot for mounting a projecting flange of a diaphragm thereto.

### BRIEF DESCRIPTION OF THE DRAWINGS

The present invention is further described in the detailed description which follows, wherein;

FIG. 1 shows a top view of a cervical traction device in accordance with the invention;

FIGS. 2a and 2b show side views of the cervical traction device in an extended and retracted position, respectively, in accordance with the invention;

FIG. 3 shows a cut-away view of the cervical traction device in accordance of the invention;

FIG. 4 shows an exploded view of the cervical traction device in accordance with the invention;

FIG. 4a and FIG. 4b (cross section) show a connection mechanism in accordance with the invention;

FIG. 5 shows a bottom view (with a bottom housing removed) of the cervical traction device in accordance with the invention;

FIG. 5a shows a top view (with cushion wedges removed) of the cervical traction device in accordance with the invention;

FIG. 6 shows a lumbar traction device in accordance with the invention;

FIG. 7 shows an exploded view of the lumbar traction device in accordance of the invention

FIG. 8 shows an exploded, perspective view of the carriage (mount) of FIG. 5, in accordance with the invention;

FIG. 9 shows a connector disk in accordance with the invention;

FIG. 10 shows a perspective view the connector disk connected to the diaphragm in accordance with the invention; and

FIG. 11 shows a cutaway view of the connector disk and diaphragm along line 11-11 of FIG. 10, in accordance with the invention.

### DETAILED DESCRIPTION OF EMBODIMENTS OF THE INVENTION

The invention is directed to a cervical and/or a lumbar traction device and more particularly, a cervical and/or a lumbar traction device having a diaphragm and connection thereto. The traction device includes a corrugated diaphragm which is structured and adapted to move a carriage in a linear fashion. The diaphragm is connected to a pump via a manifold and connection thereof. The diaphragm includes a certain spring force which is capable, upon the release of pressure therein, to move the carriage towards its original position and, in embodiments, substantially to its original position. In addition to its own spring force, a roll spring or other spring or resilient type mechanism may be used in combination with the diaphragm, to retract the carriage towards its original position. The diaphragm is devoid moving parts. The diaphragm is connected to a carriage via a novel connection mechanism. The cervical and/or a lumbar traction device, using the diaphragm and related components, is lighter and includes fewer parts than devices using pneumatic cylinders.

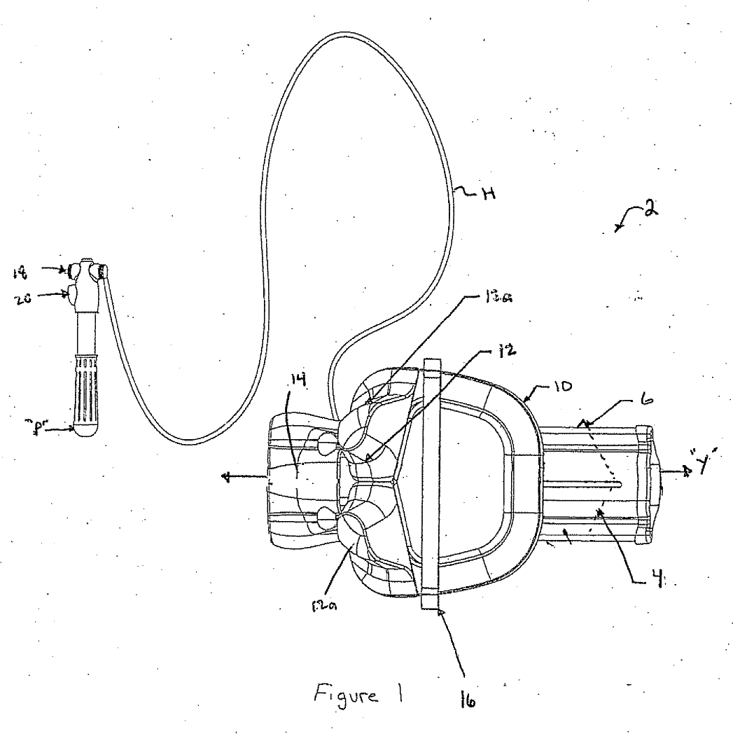

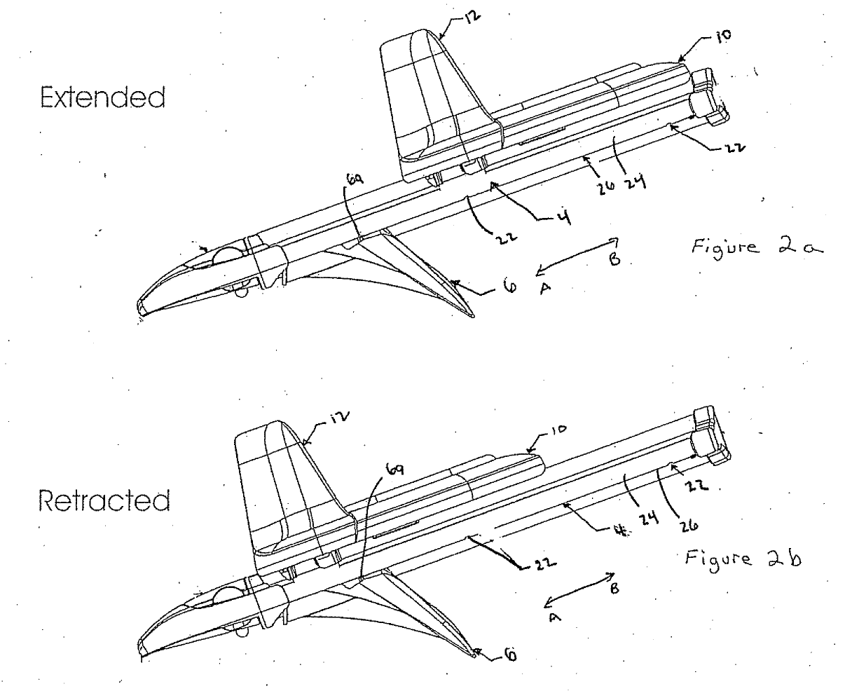

FIG. 1 shows an embodiment of the cervical traction device. The cervical traction device is generally denoted as reference numeral 2. The traction device 2 includes a stationary housing (e.g., frame) 4 having a moveable stand 6 which is structured and adapted so that several angles can be achieved relative to a flat surface, e.g., a floor or other surface where a user may use the device 2. The cervical traction device 2 further includes a movable headrest 10 having a occiput wedge system 12, which is mounted to a sliding carriage 14. A strap or other restraining device 16 for restraining a users head is attached to the movable head-rest system 10.

The occiput wedge system 12 includes separate wedges 12a that may have a concave engaging surface. The occiput wedge system 12, with the head-rest 10r may be slidably movable along a longitudinal axis “Y” of the housing 4 by a corrugated diaphragm type device (shown more clearly in FIG. 3). The occiput wedge system 12 is designed to apply a therapeutic traction force to the occipital areas on a patent's head, while the housing 4 remains stationary. This is accomplished, in one aspect of the invention, by pressure being applied by a pump P via a hose H, to the diaphragm type device. Thus, upon pressurization, the corrugated diaphragm type device will move or slide the carriage 14 and thus the headrest 10 and occiput wedge system 12. The pump P may have a manual pressure relief mechanism 18, as well as a gauge 20 to show a force created on within the diaphragm type device.

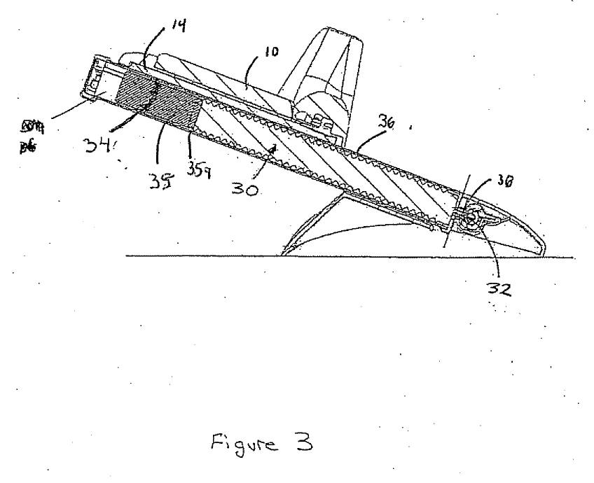

FIGS. 2a and 2b show the cervical traction device 1 positioned at an angle of, for example, 10 degrees relative to a flat surface, e.g., a floor or other surface where the user may use the device 2. It should be understood that other angles of adjustment, e.g., 15 degrees, 20 degrees, etc. with relation to the supporting surface, are also contemplated by the invention, and that the angles shown herein are provided for illustrative purposes. In the examples of FIGS. 2a and 2b, the adjustment can be accomplished via movement of the moveable stand 6 in either direction “A” or “B”, along the longitudinal axis of the device 2.

Still referring to FIGS. 2a and 2b, the housing 4 includes a guide 24 which has turned ends that form rails 26. The rails 26 are structured to accommodate the stand 6. In one embodiment, the rails 26 include a plurality of notches 22 for locking or positioning of the stand 6 at a predetermined position. The notches 22 may also be apertures or holes. In embodiments, the stand 6 may be locked into place by use of a spring arm, hook or other type of mechanism 6a that engages the notches 22 positioned along the stand 6 of the housing 4.

As discussed in greater detail below, in FIG. 2a, the traction device is in an extended position, whereas, in FIG. 2b, the traction device is in a retracted position. The extended position is achievable by pressurizing the diaphragm shown in FIG. 3. The retracted position is achievable, at least in part, by a return spring force of the diaphragm. That is, upon release of pressure, the spring force of the diaphragm is capable of moving the carriage towards its original position and, in embodiments, substantially to its original position.

FIG. 3 shows a cutaway view of the traction device in accordance with the invention. The traction device 2 includes a corrugated diaphragm type device 30 that is mounted within or mounted to the housing 4. In embodiments, the corrugated diaphragm type device 30 is mounted to a support structure 32 of the housing 4 and the movable carriage 14. In one implementation, at least one boss 34 extends from the moveable carriage 14, via a slot 36 extending substantially along a length of the housing 4, into an interior space of the housing 4, which is connected to a mounting carriage 35. In this embodiment, the corrugated diaphragm type device 30 is attached to the at least one boss 34 via a mounting carriage 35 adapted and configured to slide within the housing 4 and preferably a profile 35a of the housing 4 that is configured to house the corrugated diaphragm type device 30, Upon pressurization of the corrugated diaphragm type device 30, the corrugated diaphragm type device 30 will move the carriage 14 to apply a traction force.

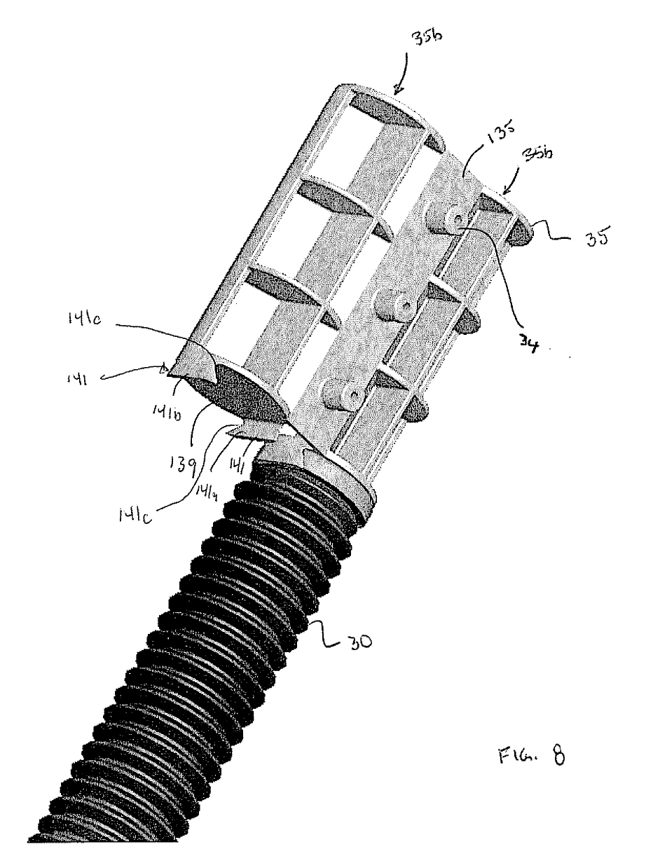

The corrugated diaphragm type device 30 is connected to an air inlet or manifold 38 provided at an end of the housing 4, proximate to the support structure 32. This connection should preferably minimize any leakage of air, at this junction. The manifold 38 is connected to one or more of the diaphragm type devices 30 and is structured to allow pressurized air from the pump “P” to pass into the corrugated diaphragm type device 30. The manifold and connection thereof is discussed in more detail with reference to FIGS. 8-10.

Still referring to FIG. 3, the corrugated diaphragm type device 30 acts as a spring, e.g., has its own spring force, to retract the carriage in both a cervical and lumbar traction system from an expanded position. In addition to its own spring force, a roll spring or other spring or resilient type mechanism may be used in combination with the diaphragm, to retract the carriage towards its original position which may be represented schematically with the diaphragm, In one exemplary illustration, the corrugated diaphragm type device 30 can be expanded to approximately 10.5 inches in the expanded position, and in one embodiment may have a starting position of about 6.5 inches in length (with an original neutral length of approximately 8 inches). It is also contemplated that other starting and expanded positions are provided by the invention. In one implementation, a stroke length of the corrugated diaphragm type device 30 is approximately 4 inches; although other stroke lengths are also contemplated by the invention.

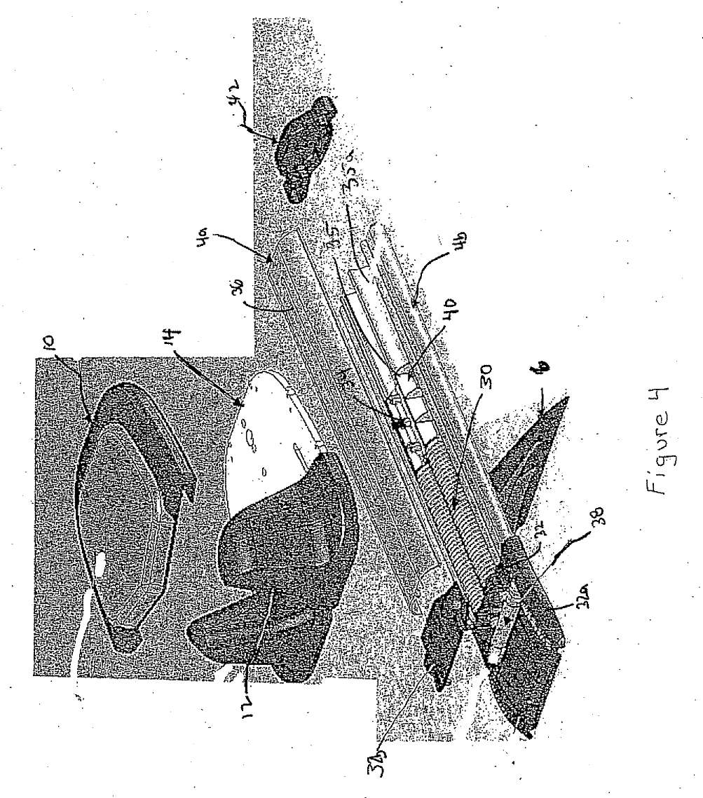

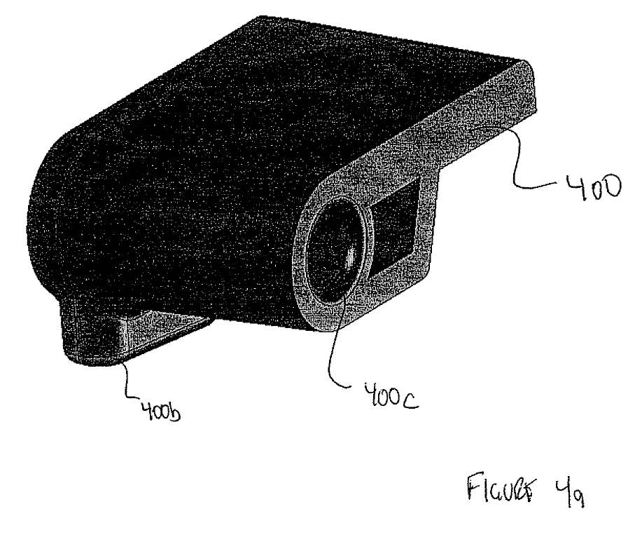

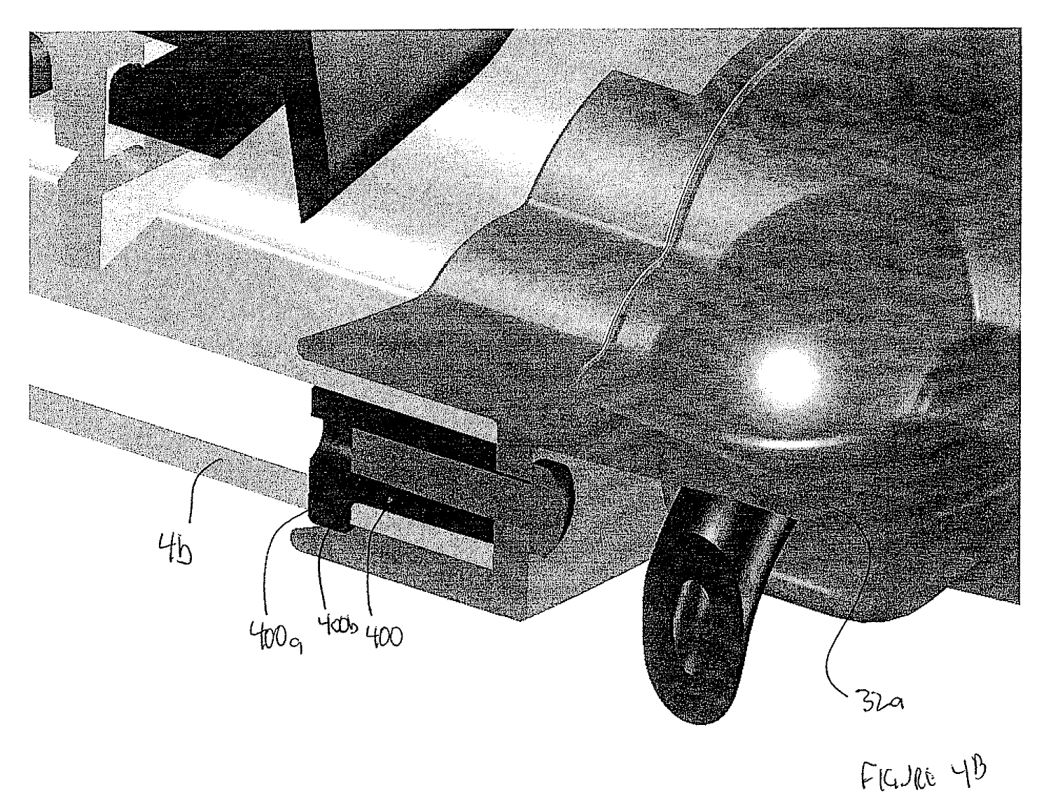

FIG. 4 shows an exploded view of the traction device in accordance with the invention. The traction device includes, for example, the headrest 10 and occiput wedge system 12 connected to the carriage 14. The carriage 14, in turn, is connected to the at least one boss 34 which extends through the slot 36 along the housing 4 and more specifically a cover plate 4a of the housing 4. The housing 4 further includes a lower frame portion 4b, which includes an interior portion 40 for housing the corrugated diaphragm type device 30.

The housing 4 further includes the support structure 32, which may include an extending portion 32a for resting on a supporting surface. A cover 32b may be used, in conjunction with the extending portion 32a, to form an interior space, which accommodates the manifold 38. An end cap 42 may be connected to the housing 4, opposing the extending portion 32a. Thus, the combination of the end cap 42, top plate 4a, bottom frame 4b and support structure forms an interior housing for the corrugated diaphragm type device 30. In alternate embodiments, the corrugated diaphragm type device 30 is mounted or attached to the mounting carriage 35 which has at least one extending boss 34 adapted and configured to slide within a guide of the housing 4 and connectable to the sliding carriage 14.

The extending portion 32a may be held in place by a nut 400 having a projection 400b, as shown in FIGS. 4a and 4b. In this implementation, the nut will be bolted via the hole 400c and the projection 400b will communicate with a slot 400a in the frame portion to prevent rotation of the extending portion 32a and to ensure a connection therebetween Alternatively, the extending portion 32a may be connected or mated with the frame by any known, conventional manner such as a screw, nut and bolt system, etc.

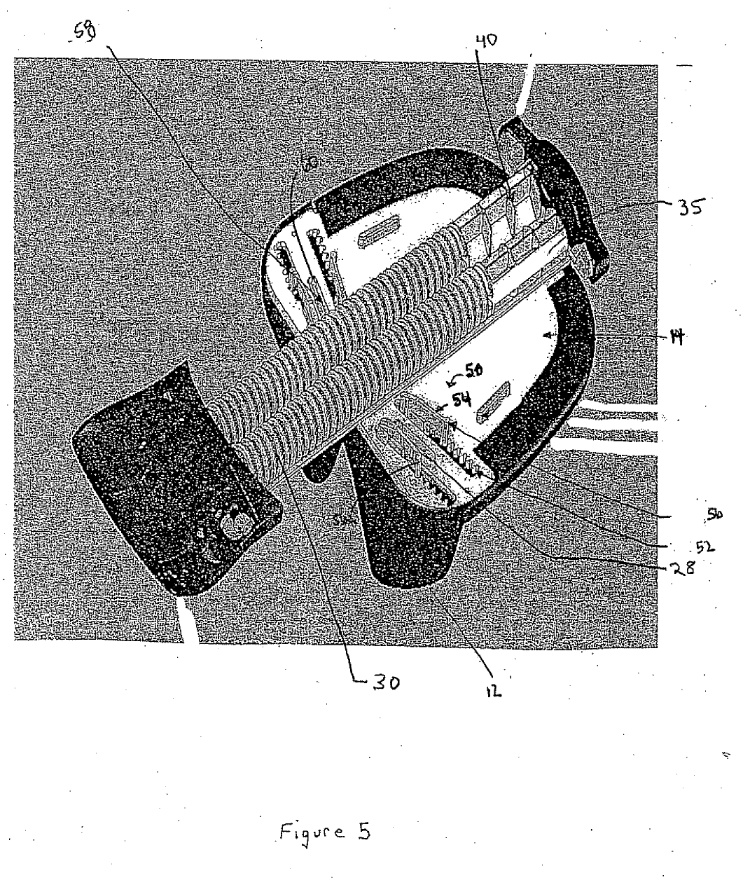

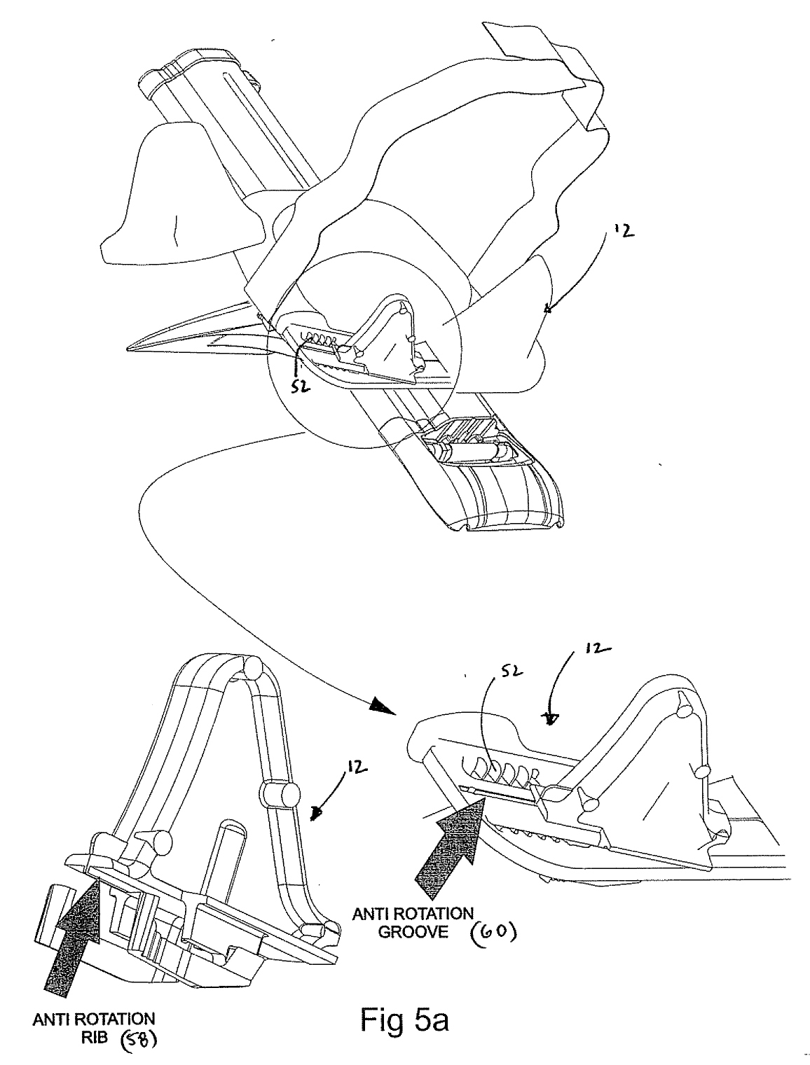

FIG. 5 shows a bottom view (with a bottom portion of the housing removed) of the traction device 2 in accordance with the invention. In this example, the wedges 12 are designed to move in a lateral direction, e.g., do not rotate. In this embodiments the wedges 12 can be adjustable by a pinch spring arm mechanism 50. The pinch spring arm mechanism 50 includes a ratchet type guide 52 in/on the moveable carriage 14. The pinch spring arm mechanism 50 further includes spring arms 54 which may engage the ratchet guide 52 at different positions via opposing protruding portions 56. The opposing protruding portions 56 have tabs 56a so that the user can squeeze them together to disengage from a ratchet portion of the ratchet guide 52, and then move the wedges in an inward position or an outward position. The movement of the wedges may be in a somewhat linear manner across the carriage 14.

Still referring to FIG. 5 and additionally FIG. 5a, the wedge system 12 may also include an anti-rotation rib 58 and corresponding slot or groove 60 for preventing rotation of the wedge 12. The slot 60 can be part of the carriage 14 and is, in one implementation, proximate to the ratchet guide 52. The rib 58 communicates with the slot 60 while allowing the wedges 12 to slide in and out. The corrugated diaphragm type device 30 is attached to the mounting carriage 35, which is adapted and configured to slide within the housing 4 and preferably within contoured portions 35a of the housing 4. The at least one boss 34, in one implementation, is attached between the sliding carriage 14 and the mounting carriage 35 such that upon pressurization of the corrugated diaphragm type device 30 will move the mounting carriage 35 which, in turn, will move the sliding carriage 14.

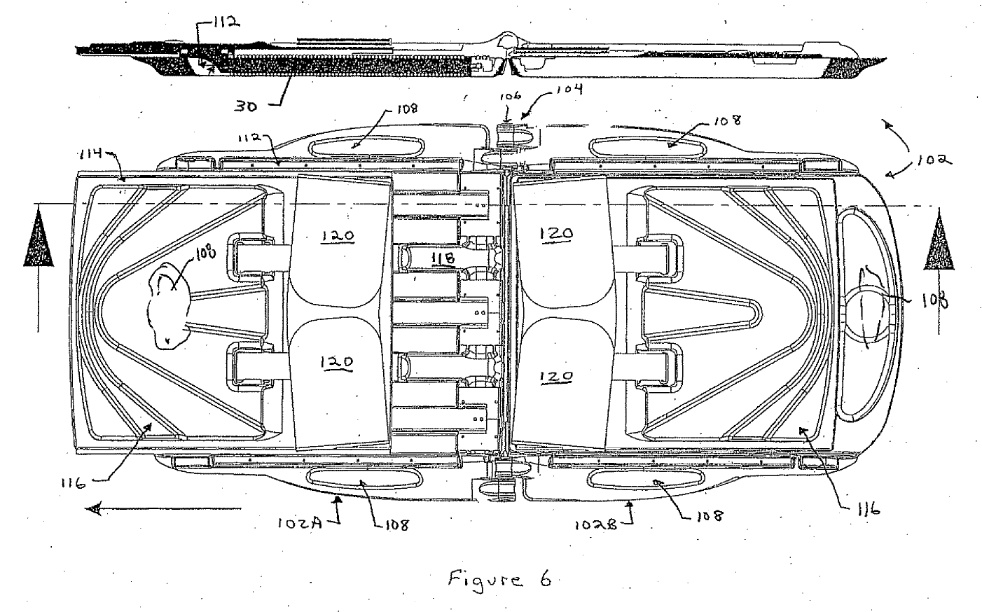

FIG. 6 shows an embodiment of a lumbar traction device 100 in accordance with the invention. Similar to the traction device shown and described with reference to FIGS. 1-5, a corrugated diaphragm type device is used to apply a traction type force. In the lumbar traction device 100, three corrugated diaphragm type devices may also be used to provide the traction force; although it is contemplated that more or less corrugated diaphragm type devices are used in accordance with the invention. Integrally molded frame members 102A and 102B are hingedly attached via a hinge type mechanism 104 (which may be an alternating type hinge mechanism having at least four parts) to form a portable, foldable device, In the closed position, for example, the frame members 102A and 102B face one another; whereas, in the open state, the frame members 102A and 102B extend along a longitudinal axis in a single plane. Thus, the design of the traction device of FIG. 6 allows for the easy closing and opening of the lumbar traction device 100 without the requirement for aligning any parts, lifting any of the parts or separating any of the parts. The lumbar traction device 100 also includes integrally formed handles 108, for example, molded on sides or the ends thereof.

Still referring to FIG. 6, the frame members 102A and 1028 are designed to house or mount thereon many of the components of the lumbar traction device 100, such as the corrugated diaphragm type device 30. By way of illustration, the lumbar traction device 100 may include guides 112 positioned on opposing sides of the frame member 102A and/or frame member 102B. The guides 112 are designed to seat thereon a slidable lumbar carriage 114. The lumbar carriage 114 is slidably moveable along the longitudinal axis of the frame 102A and 102B to provide a traction force such that, upon pressurization or release thereof, the corrugated diaphragm type device moves the slidable lumbar carriage 114 in directions “A” and “B”. In one embodiment, the lumbar carriage 114 includes a support pad 116 for the comfort of a user. A pair of belts or other restraining device 120 can be mounted to the support pad 116 and/or the frame member(s). The slidable lumbar carriage 114 may also be seated within a recessed portion 118 of the frame.

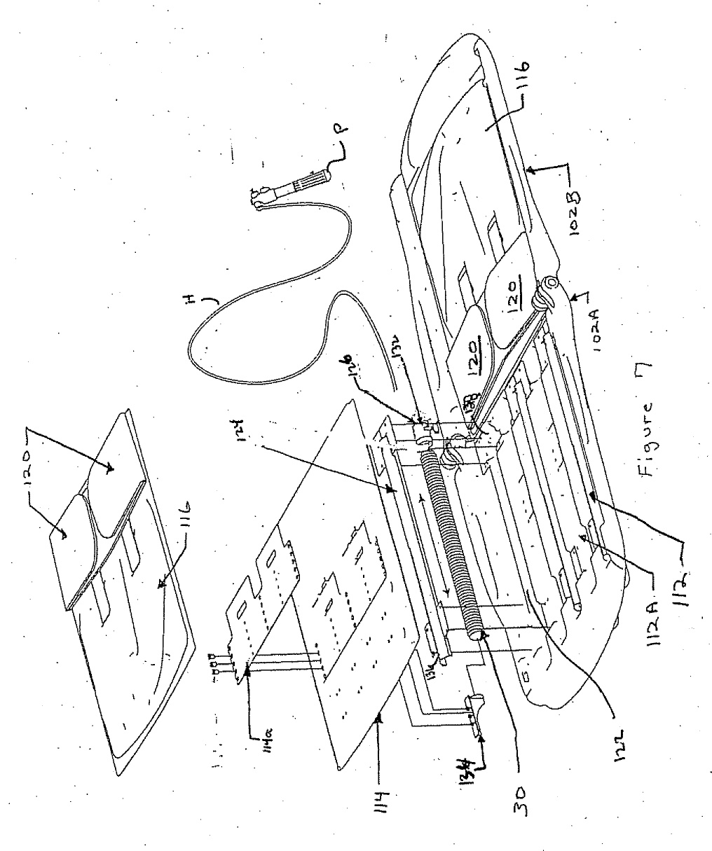

Referring to FIG. 7, the slidable lumbar carriage 114 is connected to the corrugated diaphragm type device 30 in order to provide a traction force. Much like discussed above, the corrugated diaphragm type device 30 is mounted within a housing or recess 122 formed by the frame member and a cover plate 124. In embodiments, the lumbar traction device may include two or three corrugated diaphragm type devices 30, corresponding to the number of recesses and/or the required application of traction force.

The corrugated diaphragm type device 30 is connected to the manifold 126 which, in turn, is housed inside support structure 132, which, in turn, is mounted to the support structure 128. The manifold 126 includes an air intake/exhaust 132 connected to a hose “H” of the hand pump “P”. The corrugated diaphragm type device 30 is also connected to the slidable lumbar carriage 114 via a boss 134. In one implementation, the boss 134 extends from the slidable lumbar carriage 114, via s slot or cutout 136 extending substantially along a length of the plate 124. In this embodiment, the corrugated diaphragm type device 30 is attached to the boss 134 and, upon pressurization of the corrugated diaphragm type device(s) 30, will move the slidable lumbar carriage 114 to apply a traction force. The support pad 116 may be mounted to the slidable lumbar carriage 114 via a plate 114a (and fasteners, e.g., screws, rivets, etc.)

Still referring to FIG. 7, the corrugated diaphragm type device 30 acts as a spring, e.g., has its own spring force, to retract the carriage in both a cervical and lumbar traction system from an expanded position. In addition to its own spring force, a roll spring or other spring or resilient type mechanism may be used in combination with the diaphragm, to retract the carriage towards its original position which is represented schematically with the diaphragm. In one exemplary illustration, the corrugated diaphragm type device 30 can be expanded to approximately 10.5 inches in the expanded position, and in one embodiment may have a starting position of about 6.5 inches in length. It is also contemplated that other starting and expanded positions are provided by the invention. In one implementation, a stroke length of the corrugated diaphragm type device 30 is approximately 4 inches; although other stroke lengths are also contemplated by the invention.

FIG. 8 shows an exploded, perspective view of a mounting carriage in accordance with the invention. In this view, it is shown that the mounting carriage 35 is designed and configured to accommodate two corrugated diaphragms 30 (although only one corrugated diaphragm is shown for illustrative purposes). It should be realized by those of skill in the art that the mounting carriage 35 may be designed to accommodate more or less than one corrugated diaphragm, depending on the particular application.

Still referring to FIG. 8, the mounting carriage 35 is adapted to slide within the housing 4. Accordingly, the mounting carriage 35, in one preferred embodiment, will have a cross section similar to that of the housing 4 and more preferably the contoured profile 35a of the housing 4. In the embodiment of FIG. 8, the mounting carriage 35 includes two sides 35b, each substantially identical to one another and connected to each other by a mounting plate 135. The mounting plate 135 includes at least one mounting connection or a boss 34, e.g., projections which can accommodate screws or other fastening means which are. The mounting connections 137 may be used to connect the boss 34 to the carriage 35. In embodiments, when only a single mounting plate is contemplated by the invention, the mounting plate 135 may be positioned at any surface of the mounting carriage, facing the boss.

In embodiments, the mounting carriage 35 includes end plates 139 having a cross section substantially matching the cross section of the corrugated diaphragm 30. The cross section of the mounting carriage 35, though, may also be different than that of the corrugated diaphragm 30, but should remain configured to allow mounting of the corrugated diaphragm thereon. The end plates 139 each include flanges 141, which are configured to accommodate a connector disk 143 used to connect the corrugated diaphragm 30 to the mounting carriage 35. In embodiments, the flanges 141 have at least portions which are opposing one another in order to secure the connector disk 143 thereto.

In embodiments, the flanges 141 have an inner surface 141a which is substantially flat; although, other surface contours are also contemplated by the invention. The flanges 141 may have a slightly tapered outer surface 141b and end walls 141c and, in embodiments, are mounted to or molded on the end plates 139 at a slight inward angle towards each other, In embodiments, the inward angle is such that the connector disk 143 is press fit between the flanges 141 and remains secured therebetween, as shown in FIG. 8. It should be recognized by those of skill in the art that the inward angle of the flanges 141 and the tapered end walls 141c may vary, depending on the contours and shape of the mounting disk, any of which can be obtained without undue experimentation.

In embodiments, the inward angle alone or in combination with the tapered end walls 141c can be used to securely fit the connector disk 143 between flanges 141 and hence securely fit the corrugated diaphragm to the mounting carriage 35. Alternatively, the tapered end walls 141 alone, in embodiments, can be used to securely fit the connector disk 143 between the opposing flanges 141 and hence securely fit the corrugated diaphragm to the mounting carriage 35.

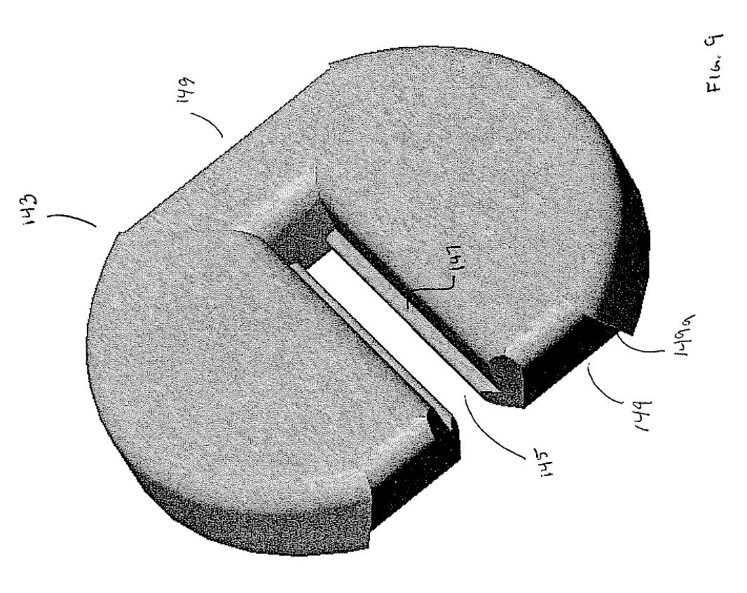

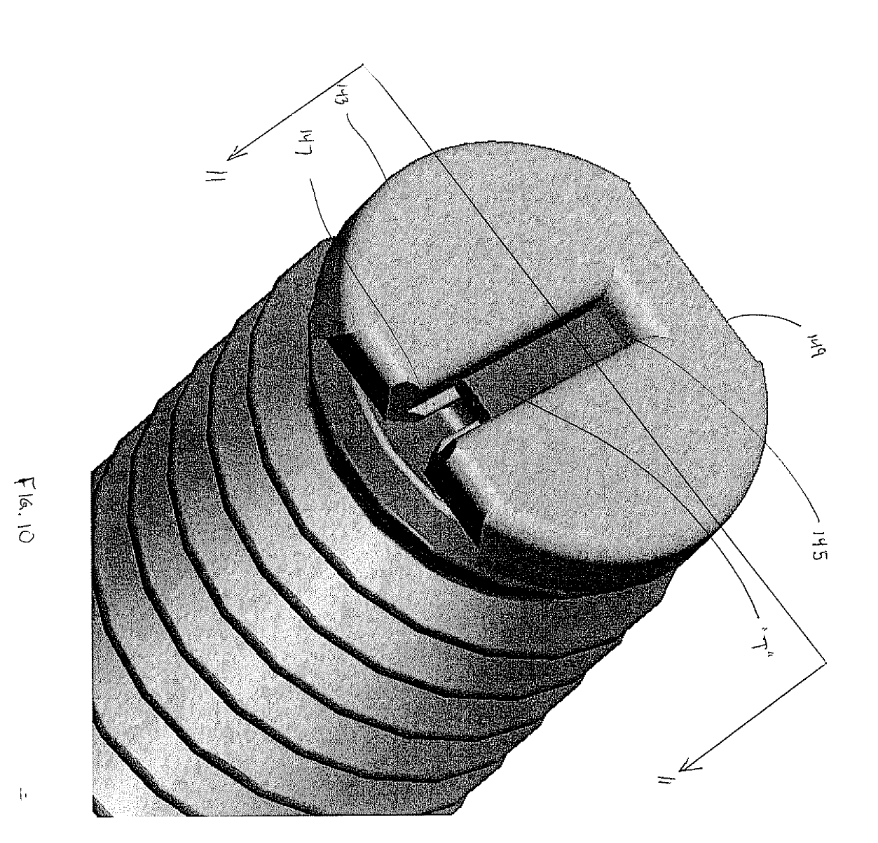

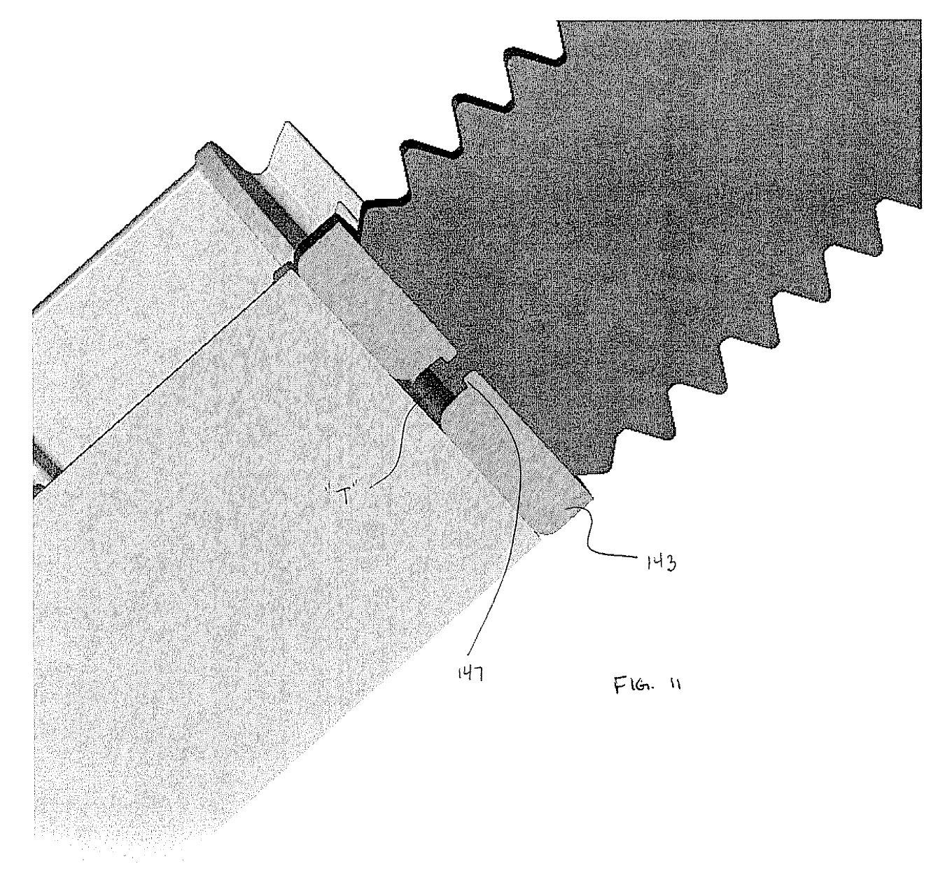

FIG. 9 shows the connector disk 143 in accordance with the invention. The connector disk 143 includes a slot 145 having a shoulder 147 running along its length. It should be recognized by those of skill in the art that the shoulder 147 does not have to run the entire length of the slot 147, and may be intermittently or singularly placed at a predetermined location along the slot 145. As discussed with reference to FIGS. 10 and 11, the slot 147 is designed and configured to mate with a “T” shaped projection extending from an end of the corrugated diaphragm 30.

The connector disk 143 further includes recessed sides 149 which are configured to mate with the inner surface 141a of the flanges 141. The recessed sides 149, in one embodiment, may have a flat surface; although other surface shapes and contours are also contemplated by the invention, all of which are configured to fit between the opposing flanges 141. In embodiments, the recessed sides 149 may be tapered to substantially match the slight inward angle of the opposing flanges 141. Additionally, in embodiments, end walls 149a of the recessed sides 149 may be tapered or angled to substantially match the taper of the tapered end walls 141c of the flanges 141. Again, the configuration of the connector disk 143 and the corresponding configuration of the flanges 141 facilitates a secure attachment between the connector disk and the mounting connector.

FIG. 10 shows a perspective view the connector disk mounted to the corrugated diaphragm in accordance with the invention. FIG. 11 shows a cutaway view of the connector disk and diaphragm along line 11-11 of FIG. 10. As shown in these views, the corrugated diaphragm includes a “T” shaped projection, extending from an end thereof. The “T” shaped projection slides within the slot 145 of the connector disk 143 and more specifically is securely held within the slot by the mating between the “T” shaped projection and the shoulder 147 of the connector disk 143. Although it is shown that the connector disk 143 has substantially a same cross section (expect for the recessed portions) as the corrugated diaphragm, it should be recognized that other shapes are also contemplated by the invention. In embodiments, for example, the cross sectional shape of the connector disk 143 matches and not be larger than that of the end plate 139 of the mounting carriage 35. It should be noted that the connection from the diaphragm to the carriage does not require a connector disk and that the carriage could have a T slot which would accommodate the “T” shaped projection of the diaphragm for connection thereto, therefore eliminating the connector disk. In this scenario, the flanges may also be eliminated.

As should now be understood in view of the above description, the corrugated diaphragm 30 is securely fitted to the connector disk 143 by way of the mating between the “T” shaped projection and slot 145. The connector disk 143 is securely fitted to the mounting carriage 35 by mating of the flanges 141 and the recessed portions 149. Thus, by virtue of these connections, the corrugated diaphragm 30 is securely attached to the mounting carriage 35, Also, by virtue of the boss being connected to the mounting carriage 35, pressurization of the corrugated diaphragm will move the moveable carriage and hence provide traction to the patient.

It should be noted that the connection between the diaphragm to the carriage does not require a connector disk. In this embodiment, the carriage could have a slot configuration which would accommodate the “T” shaped projection of the diaphragm for connection thereto, therefore eliminating the connector disk. In this scenario, the flanges may also be eliminated.

### Operation of the Traction Device

To use the traction devices, the corrugated diaphragm type device 30 is initially at atmospheric pressure. With a cervical traction device, prior to use, the patient or user can adjust the angle of the cervical traction device by adjusting the positioning of the stand. The back of the user is placed on a support surface so that the wedges cradle the patient's neck. The wedges can also be adjusted but preferably remain substantially stationary during use of the device. The restraining device may be used about the patient's head to ensure the user remains substantially stationary during treatment of cervical traction device. In the lumbar device, the patient would strap the restraining device about his/her mid section.

Once properly positioned, the patient then pumps air into the corrugated diaphragm type device by way of the pneumatic circuit described above. The patient increases the traction force by manually operating the pump or decreases the traction force by manually pressing the pressure relief mechanism. As air is pumped into the corrugated diaphragm type device, the corrugated diaphragm type device will expand and move the carriage to apply a traction force, via the mounting carriage and boss assembly. The entire structure of the cervical traction device, though, remains constant, during the application of the traction force.

When excessive pressure occurs inside of the pneumatic system, the pressure release valve will allow the release of air until the pressure is reduced to within safe operating conditions. Thus, the pressure release valve ensures that a user cannot exceed a designed traction force, thus maintaining safe use conditions of the traction device. When the pressure reaches a value less than the spring resistance, the valve automatically closes.

While the invention has been described in terms of embodiments, those skilled in the art will recognize that the invention can be practiced with modification.

What is claimed:

1. A device, comprising:
a frame;
a carriage slidable mounted on the frame;
a mounting carriage assembly; and
at least one diaphragm connectable to the carriage by the mounting carriage assembly, the at least one diaphragm is structured to elastically expand and retract, depending on an application of pressure, respectively, and to move the carriage relative to the frame, and the mounting carriage assembly mounting to the carriage.

2. The device of claim 1, wherein the mounting carriage assembly includes two sides, each substantially identical to one another and connected to each other by a mounting structure.

3. The device of claim 1, wherein the mounting carriage assembly includes a mounting structure having at least one mounting connection.

4. The device of claim 3, wherein the mounting structure connects to a boss of the carriage.

5. The device of claim 1, wherein the mounting carriage assembly includes at least one end plate having flanges.

6. The device of claim 5, wherein the flanges are opposing flanges.

7. The device of claim 5, wherein the flanges are angled toward each other.

8. The device of claim 7, wherein the flanges include tapered end walls.

9. The device of claim 8, further comprising a connecting disk mountable between the flanges.

10. The device of claim 9, wherein the connecting disk includes a slot having a shoulder and portions which are configured to be mountable between the flanges.

11. The device of claim 10, wherein the shoulder runs intermittently along a length of the slot.

12. The device of claim 10, wherein the portions are recessed portions which match an angle of the flanges.

13. The device of claim 10, wherein the at least one diaphragm includes a “T” shaped projection mating with the slot and shoulder of the connecting disk.

14. The device of claim 1, wherein the mounting carriage assembly comprises:
inwardly angled flanges and tapered end walls; and
a connector disk having at least one recess which interfaces with at least one of the flanges, the connector disk further having a slot which corresponds with a projection extending from the at least one diaphragm.

15. The device of claim 14, wherein the at least one recess is two recesses configured to substantially match an angle of the inwardly angled flanges.

16. The device of claim 1, wherein the mounting carriage assembly includes at least one end plate having a slot therein, and the at least one diaphragm includes a projection which mates with the slot for connection thereto.

17. The device of claim 14, wherein the projection is a T shaped projection which is slidably mounted within the slot of the connector disk and is securely held therein by a shoulder of the slot.

18. The device according to claim 1, wherein the device is a cervical traction device or a lumbar traction system.

19. A device comprising a frame for accommodating at least one corrugated diaphragm device, the at least one corrugated diaphragm device having a retractable spring force, wherein upon release of pressure within the at least one corrugated diaphragm device, the at least one corrugated diaphragm device retracts the moveable carriage towards an initial position, a connecting assembly including at least a connecting mechanism connectable between a body of the connecting assembly and the at least one corrugated diaphragm device and a mounting structure for connecting to the moveable carriage.

20. The device of claim 19, wherein the connecting assembly includes two sides, each substantially identical to one another and connected to each other by the mounting structure.

21. The device of claim 20, wherein the mounting structure connects to a boss of the carriage.

22. The device of claim 19, wherein the connecting mechanism includes at least one end plate having opposing flanges.

23. The device of claim 22, wherein the opposing flanges are angled toward each other.

24. The device of claim 23, wherein the opposing flanges include tapered end walls.

25. The device of claim 23, wherein the connection mechanism includes a connecting disk having a slot with a shoulder, the connecting disk is mountable between the opposing flanges.

26. The device of claim 25, wherein the connecting disk includes at least one recessed portion which is configured to interface with at least one flange of the flanges.

27. The device of claim 26, wherein the at least one recessed portion includes two recessed portions which match mate with the opposing flanges.

28. The device of claim 19, wherein the at least one corrugated diaphragm device includes a “T” projection configured to mate with a slot and shoulder of the connection mechanism.

29. The device of claim 19, wherein the connecting assembly comprises:
inwardly angled flanges and tapered end walls, and
a connector disk having recesses which mount between the flanges and remains secured therebetween, the connector disk further having a slot which it mated with a projection extending from the at least one diaphragm.

30. The device according to claim 19, wherein the device is a cervical traction device or a lumbar traction system.

31. A device comprising:
a mounting carriage assembly, comprising:
a frame having at least one mounting connection and at least one end plate, the at least one end plate including opposing flanges; and
a connecting disk mountable between the opposing flanges, the connecting disk includes a slot having a shoulder.

32. The device of claim 31, wherein the opposing flanges are angled toward each other.

33. The device of claim 32, wherein the opposing flanges include tapered end walls.

34. The device of claim 31, wherein the shoulder runs intermittently along a length of the slot.

35. The device of claim 31, wherein the connecting disk includes recessed portions which are configured to be mountable between the opposing flanges, the recessed portions are configured to match an angle of the opposing flanges.

36. The device of claim 31, further comprising a traction device comprising:
a frame;
a carriage slidable mounted on the frame; and
at least one diaphragm connectable to the carriage via the mounting carriage assembly, the at least one diaphragm is structured to elastically expand and retract, depending on an application of pressure, respectively, and to move the carriage relative to the frame, and the at least one diaphragm is connectable to the connecting disk.

37. The device of claim 36, wherein the at least one diaphragm includes a “T” shaped projection which mates with the slot and shoulder of the connecting disk.

38. A device comprising a mounting carriage assembly including a frame having at least one mounting connection and at least one end plate, the at least one end plate includes a slot for mounting a projecting flange of a diaphragm thereto.

39. The device of claim 38, further comprising a traction device comprising:
a frame;
a carriage slidable mounted on the frame; and
at least one diaphragm connectable to the carriage via a slot mount within the mounting carriage assembly, the at least one diaphragm is structured to elastically expand and retract, depending on an application of pressure, respectively, and to move the carriage along the frame.
